(19) United States
(12) Patent Application Publication
(10) Pub. No.: US 2024/0402156 A1
(43) Pub. Date: Dec. 5, 2024

(54) Nanopore System to Assess Properties of Viral Particles

(71) Applicant: Northeastern University, Boston, MA (US)

(72) Inventors: Meni Wanunu, Sharon, MA (US); Sara Rouhanifard, Chestnut Hill, MA (US)

(21) Appl. No.: 18/262,659

(22) PCT Filed: Jan. 25, 2022
(86) PCT No.: PCT/US2022/013765

§ 371 Date: Jul. 24, 2023

Related U.S. Application Data
(60) Provisional application No. 63/141,270, filed on Jan. 25, 2021.

(51) Int. Cl. G01N 33/50; B01L 3/00; C12M 1/34

(52) U.S. Cl. CPC G01N 33/5091; B01L 3/502715; C12M 41/46; G01N 33/5023; B01L 2300/0645; G01N 2333/005

(57) ABSTRACT

A system and method for assessing properties of particles, the system comprising: a first structure defining an interior cavity, configured to contain an interior cavity fluid, and a barrier separating the interior cavity from an exterior cavity configured to contain an exterior cavity fluid, the barrier defining a nanopore therethrough fluidically coupling the interior and exterior cavities, the first structure further comprising at least one feature configured to interface with at least one complementary feature of a second structure that defines the exterior cavity; and electrodes configured to energize and apply a voltage gradient across the nanopore between the interior cavity and exterior cavity, the voltage gradient of sufficient magnitude to induce particles to migrate via the nanopore between the interior cavity and the exterior cavity and produce an electronic signature representative of at least one property of the particles.

### RELATED APPLICATION

This application claims priority to U.S. Application No. 63/141,270, filed on Jan. 25, 2021. The entire teachings of the above application are incorporated herein by reference.

### BACKGROUND

Direct and sensitive quantification and characterization of viral and other particles in a given sample is a significant challenge for virology, both in research and clinical settings. Viral vectors are critical tools for delivering DNA into cells for a variety of promising new gene therapies, including CAR T-cell therapeutics, CRISPR therapeutics, and vector-based vaccine delivery. A major hurdle for the developers of therapies relying on viral vector delivery lies in precisely measuring counts of infectious virions in addition to quantifying the full composition of matter in the viral product. Currently, even the best existing methods for counting viruses are limited leading to significant assay variability and downstream interpretation of clinical trial data.

There are five main types of viral quantification methods: 1. qRT-PCR that detects and quantifies a viral genome, 2. Transmission electron microscopy (TEM) that shows viral particles with their coat proteins, 3. Enzyme-linked immunoassay (ELISA) that utilizes antibodies to detect the presence of a viral antigen, 4. Cell infectivity assay that either quantifies the number of viral plaques formed or the fluorescence produced from cells infected with a fluorescent transgene, and 5. Median tissue culture infectious dose (TCID50) assay that quantifies the amount of virus required to kill 50% of the infected host. Each of these methods is limited because each is an indirect measurement of the total number of particles in a given sample and requires time and specialized equipment to perform. Many existing methods also have limitations when counting viral particles independent of infected cells.

Batch-to-batch variability in virions can also cause outright study failure because non-infectious virions compete with cellular target receptors. Furthermore, the preferred infectivity assays performed in virology labs are highly dependent on the specific cell line used for viral infection. Natural viruses are not engineered to contain a fluorescent transgene for easy identification, and not all viral particles will contain the viral genome. Thus, there remains a major gap in technology for quantifying virus particles in a given sample that measures the total number of particles as well as the infectivity and replication efficiency of the given particles. In particular, gene therapy and vaccine development industries are suffering a great deal due to the use of inaccurate existing methods for quantifying the composition of matter in their viral vector products. Therefore, a need exists for an ability to obtain precise measurements of viral and other particles, which would represent not only a technical advance but would also increase the global robustness of viral delivery assays. In some embodiments, the reference standard and the test sample are from the same subject.

The particles or viral particles may be able to bind to cell receptors. Some embodiments of the invention may further comprise applying single-molecule RNA fluorescence in situ hybridization (smFISH) to determine a percentage of cells infected with the particles or viral particles. Additionally, embodiments of the invention may include imaging a fluorescent reporter delivered to the test sample.

### SUMMARY

An example embodiment of the invention enables methods, and associated systems, for simultaneously assessing an absolute viral particle count as well as the functional titer and replication efficiency of viral particles in a given sample. Embodiments of the invention may employ a combination of (i) nanopore technology that can discriminate viral particles based on biophysical properties and (ii) single-molecule RNA fluorescence in situ hybridization (smFISH) that can discriminate down to single molecules of viral RNA in infected cells. Embodiments of the invention may provide a direct way to determine a concentration of packaged viruses in a sample instead of the indirect metrics (such as plaque-forming units, or PFU) utilized in the existing technology. These example embodiments provide a solution to the problem of direct quantification of infective virus particle count and also enable single-virus precision in quantification of infectivity. These example embodiments also allow for measuring of size distribution and enable precise virus quantification by multiple, independent measurements. In addition, embodiments of the invention can be easily integrated into a standard laboratory environment without the need for large equipment or dedicated space. In fact, embodiments of the invention can be utilized with standard cell culture chambers.

A system for assessing properties of particles is disclosed herein. The system comprises a first structure defining an interior cavity, configured to contain an interior cavity fluid, and a barrier separating the interior cavity from an exterior cavity configured to contain an exterior cavity fluid, the barrier defining a nanopore therethrough fluidically coupling the interior and exterior cavities, the first structure further comprising at least one feature configured to interface with at least one complementary feature of a second structure that defines the exterior cavity. The system also includes electrodes configured to energize and apply a voltage gradient across the nanopore between the interior cavity and exterior cavity, the voltage gradient of sufficient magnitude to induce particles to migrate via the nanopore between the interior cavity and the exterior cavity and produce an electronic signature representative of at least one property of the particles.

The particles assessed by the system may be microbes, such as but not limited to virus particles, bacteria, fungi and nanosomes. The system may further include a detector configured to observe the electronic signature and a processor configured to determine a number of particles that migrated via the nanopore based on the electronic signature observed. The system may additionally comprise the second structure, wherein the second structure defines a wall that is separated from the barrier with the exterior cavity fluid therebetween while the at least one feature of the first structure is interfaced with the at least one complementary feature of the second structure.

In such embodiments, the system may further comprise an imager configured to determine a number of cells in the second structure to which at least a subset of the particles associated themselves, the imager further configured to provide a representation of the number of cells determined to the processor. The particles may be viral particles and the processor is further configured to determine infectivity of the particles based on a ratio of the number of particles that migrated via the nanopore and the representation of the number of cells determined to have at least one viral particle associated therewith.

The imager may be an epifluorescence microscope and the particles include a fluorescent reporter detectable by the epifluorescence microscope. Alternatively, the imager may be configured to detect nucleic acid of the particles or expression products if the particles.

The particles may be vectors that may provide nucleic acid to the cells. The vectors may provide nucleic acid to the cells. The vectors may be viral vectors. The processor may also be further configured to determine a particle size distribution.

In certain embodiments the second structure may also define a micro fluidic channel in fluidic communication with the nanopore. The second structure may be a cell culture chamber and the system may further include a cover configured to removably couple with the second structure by interfacing with the least one complementary feature of the second structure and the first structure may be a lid, substitutable for the cover.

Embodiments of the system may include a detector configured to observe the electronic signature and a processor configured to determine a number of particles that migrated via the nanopore based on the observed electronic signature and to assess a number of particles that are less than about 1 micrometer in size.

A method for assessing properties of particles is also disclosed herein. The method comprises interfacing a first structure to a second structure by way of coupling at least one mechanical complementary feature of the first structure to at least one mechanical complementary feature of the second structure, the first structure defining an interior cavity, the first structure further defining a barrier separating the interior cavity from an exterior cavity configured to contain an exterior cavity fluid, the barrier containing a channel structure defining a nanopore therethrough fluidically coupling the interior and exterior cavities, the first structure further comprising at least one feature defining a shape configured to interface with at least one complementary feature of a second structure that defines the exterior cavity. The method also includes applying a voltage gradient across the nanopore between the interior cavity and exterior cavity by energizing electrodes, the voltage gradient being of sufficient magnitude to induce particles to migrate via the nanopore between the interior cavity fluid and the exterior cavity fluid and to enable observing of an electronic signature representative of at least one property of the particles produced by passing through the nanopore.

The particles may be microbes. The method may further include adding an interior cavity fluid containing the particles to the interior cavity.

Some embodiments of the method may further comprise observing, using a detector, the electronic signature to produce an observed electronic signature and determining, using a processor, a number of particles that migrated via the nanopore based on the observed electronic signature. In such embodiments, the method may also include determining, with an imager, a number of cells in the exterior cavity of the second structure, with which at least one particle associated and determining, using a processor, the infectivity of the particles based on a ratio of the number of particles that migrated via the nanopore and the number of cells with which at least one particle associated.

Also disclosed herein is a method for assessing efficiency of particles in a sample comprising cells. This method comprises applying a voltage gradient across a nanopore to induce particles to migrate from a first cavity to a second cavity via the nanopore to produce an electronic signature representative of each particle during migration, the second cavity containing cell. The method also includes counting a number of particles based on the electronic signature to create a total particle count of migrated particles. The method concludes by determining a number of cells with which at least one of the migrated particles associated itself to produce a total associated cell count in the second cavity determining efficiency of the particles based on a ratio of the total associated cell count and the total particle count.

Also disclosed herein is a method of assessing the efficacy of an anti-viral agent. The method includes contacting a sample of cells with an anti-viral agent to produce a test sample and exposing the test sample to a specific number of viral particles by controlling a flow of viral particles to the test sample via an electric field applied to a nanopore, the viral particle originating from a first cavity defined by first structure and the test sample is in a second cavity defined by a second structure, the first cavity and the second cavity in fluid communication via the nanopore. The method then includes determining a percentage of cells in the test sample infected by the viral particles in the second cavity and comparing the percentage of cells in the test sample infected by the viral particles to a reference standard to determine efficacy of the anti-viral agent.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is a front view illustration of a structure that includes a nanopore utilized by embodiments of the invention.

FIG. 1B is a graph of current vs. time of the circuit created by the structure shown in FIG. 1A.

FIG. 2 is an illustration of an “odds” and “evens” single-molecule FISH labeling technique.

FIG. 3A is a top view of an illustration of a cell chamber containing cells.

FIG. 3B is a top view image of HeLa cells infected with lentivirus.

FIG. 3C is a closeup view of an infected cell in FIG. 3B.

FIG. 4A is a top view of a currently existing cell culture tray.

FIG. 4B is a front view of a cell culture tray with a lid modification, according to an example embodiment of the invention.

FIG. 5A is a rendering of a structure that includes a nanopore utilized by embodiments of the invention.

FIG. 5B is a picture of 3D printed structures that include a nanopore utilized by embodiments of the invention.

FIG. 5C is an isometric diagram of a lid modified to include a nanopore and configured to interface with a set of cell culture chambers utilized by embodiments of the invention.

FIG. 6 is a flowchart of a method for determining the infectivity of viral particles according to an embodiment of the invention.

### DETAILED DESCRIPTION

A description of example embodiments follows.

Viral vectors are critical tools, both for gene delivery into cells (e.g., Car T-cells, CAR-NK cells, and CRISPR gene editing) and for vaccine delivery. Retroviral vectors, typically lentiviruses (a single-stranded RNA virus), are a preferred method for ex vivo genetic modification of T cells for CAR T-cell therapies of certain cancers. Briefly, T cells are extracted from the patient's blood and transduced with a lentivirus vector bearing an expression cassette for the chimeric antigen receptor (CAR, a synthetic antigen-binding protein that initiates signal transduction upon binding), thus generating T cells that can recognize and destroy tumor cells. Ex vivo genetic manipulation of patient cells with lentivirus is common, but direct use in vivo is also being tested. Lentivirus vectors are being used for gene editing, e.g., CRISPR/Cas gene editing systems, but adeno-associated viruses (AAV; a single-stranded DNA virus) are currently the most widely used for CRISPR/Cas gene delivery in vivo. AAV vectors have recently been employed as vaccine vectors due to their high immunogenicity, stimulating responses to a variety of antigens in vivo. Recent and famous examples of this mode of vaccine delivery are the AAV-based vaccinations for SARS CoV-2 that are distributed by large pharmaceutical corporations.

A major hurdle in working with viral vectors for therapeutic use or more traditional diagnostic/treatment use lies in measuring counts of infectious virions precisely in addition to quantifying a full composition of matter in the viral product. Viruses can be detected and measured by either functional or physical assays. Functional titration assays can be used to measure the infectious titer (i.e., how many particles are capable of infecting cells). These measurements are typically collected via plaque assay, viral transduction assay, and end-point titration methods; however, these methods do not detect noninfectious particles. Noninfectious particles include virions lacking genome or virions with damaged cell entry proteins that can interfere with the delivery of gene cargo and skew the dosing.

Quantification of noninfectious particles is useful for assessing the quality of a virus preparation. Physical assays such as transmission electron microscopy (TEM); can obtain a total particle count, however, this method is laborious and expensive to perform, and therefore, infrequently used. Quantitative real-time polymerase chain reaction (PCR) is a widely used physical method for determining genome containing units, but it is important to note that nucleic acid detected does not always correspond to the infectious virus and does not provide information about the noninfectious viruses.

The titer of a virus is often calculated in plaque-forming units (PFU) per mL although this can also be expressed as transducing units (TU) per mL depending on the assay used. The total particle-to-PFU ratio is defined as the total number of particles divided by the PFUs. For many viruses, the particle-to-PFU ratio can be extremely high and variable. For example, the particle-to-PFU ratio for Varicella-zoster virus is 40,000, while for adenovirus it can range from 20-100. For HIV-1, the ratios have even more variability ranging from 1-107. In these cases, the properties measured biochemically may not be directly attributed to those of the infectious particles, thus complicating the quality control and assessment of performance for gene therapies in vivo. The measurement of how much virus actually infects a target cell for lentivirus and AAV is expressed in the form of transducing units per mL.

The disclosed methods and systems herein enable the characterization and quantification of the infectious and non-infectious particles, at least by calculating a ratio of total particles to cells infected or associated with particles. As defined herein, the term “particle” refers to a microbe, such as but not limited to a viral particle, bacteria, fungus, or fragments thereof, or a nanosome, e.g., a liposome. As defined herein, the term “associated” and the like means that a particle interacts, contacts or enters a cell, such as by fusion, endocytosis, or penetration (e.g., injection of a viral capsule or genome into a host cell). While embodiments described herein may reference viral particles and infections, a person of ordinary skill would understand that these embodiments may also utilize non-viral particles, such as nanosomes, bacteria, or fungus that associate with cells.

Because of this innovation, the process of generating viruses can be optimized to maximize their infectious payload. Embodiments of the invention enable quantification and/or qualification of the delivery of vectors that provide nucleic acid to cells. These vectors may be, e.g., viruses or liposomes for providing modified and/or unmodified nucleic acid (e.g., deoxyribonucleic acid (DNA) or Ribonucleic acid (RNA)) to cells for therapeutic delivery to cells. The vectors may be used for therapeutic delivery, and may deliver therapies, but are not limited to chimeric antigen receptor (CAR) therapeutics (e.g., Car T-cells, CAR-NK cells, TCR, etc.), gene editing (e.g., CRISPR therapeutics), or vaccine delivery. The vectors may be viral vectors, for non-limiting example natural viruses, engineered viruses, for example at least one of lentivirus vectors, Adeno-associated virus (AAV) vectors, or herpes simplex virus (HSV)-based vectors.

In some embodiments the particles are whole viruses or fragments thereof, such as spike proteins.

In some embodiments, the particles (e.g., viruses) are natural. In some embodiments, the particles are engineered (e.g., genetically engineered).

In some embodiments, the particles are lipid nanoparticles.

In some embodiments, the vectors deliver biomolecules, such as glycans, matrices or metabolites. In some embodiments, the virus is to treat subjects in need of a protein, for example, an enzyme, e.g., for enzyme replacement therapy.

In some embodiments, an activity assay is used to determine whether a protein, e.g., an enzyme, is functional.

In some embodiments, the particles are coated, e.g., to reduce or prevent aggregation.

In some embodiments, expression products from the nucleic acid (such as proteins, e.g., human proteins or microbial proteins) are detected and/or measured.

Precise measurements of virions or other particles with embodiments of the invention represent not only a technical advance but will increase the global robustness and quality of viral delivery assays. Embodiments of the invention may also provide a useful tool for development of diagnostics by creating a gold standard for viral or other particle quantification so that counts can be compared between different experiments and laboratories. Embodiments of the invention also provide improved methods and systems for determining viral infectivity and the effects of anti-viral agents on infectivity.

An embodiment of the invention uses nanopore technology that can identify and quantify the number of viral particles that migrate or transport through a nanopore. This provides an accurate and exact count of viral particles in a sample that the viral particles migrate via the nanopore from fluid in a cavity on a first side of the nanopore 112 to fluid on the other side of the nanopore 112. Additionally, the embodiment can provide a measurement of viral concentration of in a sample from which the viral particles migrate via the nanopore based on the rate of migration. Even, alone, this ability to count viral particles directly and determine both the concentration and number of particles is a significant improvement over existing techniques. This improvement can be combined with methods, such as but not limited to single-molecule RNA fluorescence in situ hybridization (smFISH), that are able to determine the number of infected cells. Therefore, embodiments of the invention are able to determine the total viral count of the sample, using the nanopore, and the count of infective viral particles, using smFISH or an equivalent method. Then using these determined counts, embodiments of the invention can also calculate the number non-infective viral particles and viral particles infectivity. In addition, embodiments of the invention can also use a functional assay (e.g., a readout, such as an enzymatic readout, measuring a functional titer) to determine the amount of activity induced by the viral particles, and, optionally, determine the percentage of active particles in combination with the determined counts.

Embodiments of the invention may use on or more processors configured to calculate the total viral count of the sample and the count of infective viral particles based on inputs received from detectors, imagers, and other sources. These processors may also be configured to calculate the number non-infective viral particles and viral particles infectivity. The utilized processors may be local to the rest of the embodiment of the invention or remote and accessed through a cloud computing service or software.

Example embodiments of the invention provide a novel method for simultaneously or sequentially assessing an absolute viral particle count as well as a functional titer and replication efficiency of viral particles in a given sample. The principle is based on a combination of nanopore technology that can discriminate viral particles based on biophysical properties, and single-molecule RNA fluorescence in situ hybridization (smFISH) that can discriminate down to single molecules of viral RNA in infected cells. The method utilizes a modified chambered coverslip with a removable lid that contains a nanopore chip into which the viral sample is injected, and live, adherent cells plated on the bottom. The viral sample enters the nanopore for direct detection and quantification that will give absolute counts of viral particles; however, this value does not provide the infectivity. The count also does not discriminate between particles that have the functional surface proteins and the functional viral genome. After the viral particles pass through the nanopore and produce a value representing the total number of particles, they will enter a chamber containing cell culture media and cells on the bottom for a viral transduction. The cells can be directly imaged from the bottom of the coverglass using inverted, epifluorescence microscopy to quantify fluorescent cells (if using a fluorescent reporter in the virus), or smFISH may be performed on them directly to detect viral RNA particles in infected cells. Further, the number of viral particles entering the cells may be controlled using the nanopore, and the viral replication can be quantified (i.e., a single particle passes through the pore and infects a cell—how many RNA molecules are produced from that infection in a given period of time. Viral particle infectivity may be measured by comparing the total number of particles with the number of cells infected.

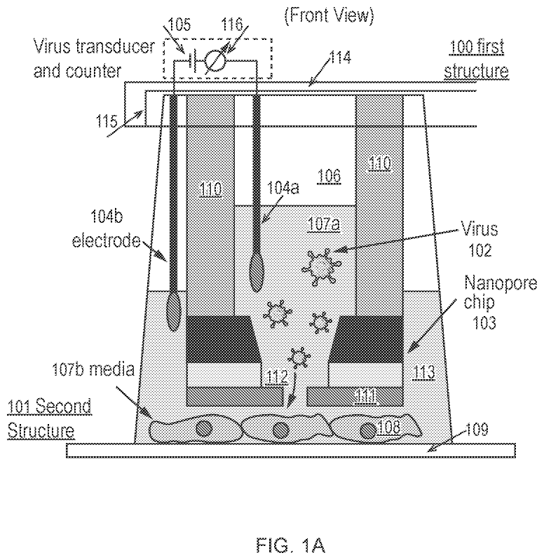

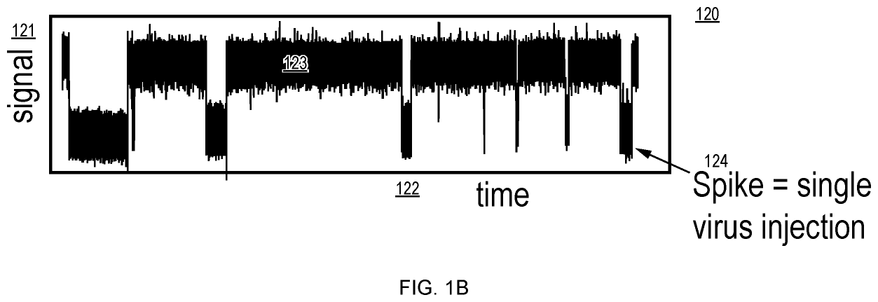

FIG. 1A is a diagram of a first structure 100 that includes a nanopore device utilized by embodiments of the invention. The structure 100 defines a cavity 106, also referred to herein as an interior cavity 106, in which fluid and viral particles 102 can be deposited and contained. The cavity 106 is bounded on two sides by walls 110. The structure 100 may include a top 114 bounding the cavity 106. The top 114 may be removable to enable a user to deposit a fluid in the cavity 106. The structure 100 further includes a nanopore chip 103. The nanopore chip 103 may be a silicon chip that contains free-standing silicon nitride membranes. In such embodiments, these nanopore chips 103 may be fabricated through a two-day Si/SiO₂/SiN, wafer processing cycle that yields over 250 individual chips per wafer. The nanopore chip 103 includes a barrier 111 defining a portion of the cavity 106. The barrier 111 includes a pore or nanopore 112 through which viral particles 102 may migrate. The nanopore 112 may be of sufficient size to allow the transport of AAV particles (20-25 nm in diameter) and lentivirus particles (80-100 nm in diameter). In some embodiments, the nanopore 112 may be 1 micron in diameter. Nanopore(s) 112 may be fabricated through the free-standing silicon nitride membranes, acting as barrier 111, using Focused Ion Beam milling or other techniques to control their diameter. The diameter of the nanopore 112 can be controlled to be between one times (1×) and four times (4×) the average diameter of the viral particle 102 to be examined.

The surfaces of the nanopore chip 103 and the barrier 111 defining the nanopore 112 may be modified to reduce clogging and achieve continuous translocation through the pore for most virus types. Surface thickness, composition, and charge may be fine-tuned by chemically modifying the fabricated pores using Atomic Layer Deposition and/or PEG-Silane surface chemistry with hydroxy, methoxy, amino, and various other functional groups. For example, silane-based surface modification significantly reduces permanent clogging of nanopores by particles or contaminants, and therefore increases the lifetime of the nanopore 112. In some embodiments, the surface(s) may be coated with alumina through Atomic Layer Deposition, and passivation using polyphosphonates allow for in-house assembly and dry storage.

The first structure 100 may include a at least one feature 115 configured to interface with at least one complimentary feature of the second structure 101. The feature 115 allows for the first structure 100 to be removably coupled with the second structure 101. In the embodiment shown in FIG. 1, the first structure 100 is a lid for the second structure 101, which serves as a cell culture chamber. When the first structure 100 is coupled with the second structure 101, the nanopore 112 places the cavity 106 in fluid communication with an exterior cavity 113, defined by the second structure 101, permitting migration of the viral particles 102 from the interior cavity 106 to the exterior cavity 113.

Migration of the viral particles 102 would be rare or possibly nonexistent without the application of outside forces. Therefore, embodiments of the invention include pair of electrodes 104a, 104b. In the embodiment shown in FIG. 1, the electrodes 104a, 104b are attached to the first structure 100, but in alterative embodiments, the electrodes 104a, 104bs may be part of the second structure 101 or a different structure altogether.

Electrodes 104a, 104b are connected to a power source 105 and that is able to energize the electrodes 104a, 104b. The electrodes 104a, 104b are placed in different cavities 106 and 113; when energized the electrodes 104a, 104b create an electrical gradient between the cavities 106 and 113 and across the nanopore 112 (i.e., from opening to opening of the nanopore 112). This electrical gradient will provide a force that induces the migration of the viral particles 102 from the interior cavity 106, through the nanopore 112 and into the exterior cavity 113. The electrical gradient can also be reversed to stop or prevent migration of the viral particles 102 from the interior cavity 106, through the nanopore 112 and into the exterior cavity 113. In alternative embodiments, the viral particles may migrate from the exterior cavity 113 to the interior cavity 106 if the electrical gradient is applied in the opposite direction.

When the electrodes 104a, 104b are energized, a circuit is created with the electrodes 104a, 104b and the power, or voltage, source 105. The resistance in the circuit is primarily provided by the first structure 100, since it is placed between the electrodes 104a, 104b and, specifically, the nanopore 112 defined by the barrier 111. The fluids 107a, and 107b in the cavities 106 and 113 are conductive and provide minimal resistance to the movement of ions in the circuit. If a viral particle 112 is in the nanopore 112, during the migration process, the circuit's resistance changes. Due to Ohms law, this change in resistance results in a change in current flowing in the circuit. The current can be monitored and measured by an ammeter 116. In the embodiment shown in FIG. 1, the ammeter 116 is integrated with the power source 105. In alternative embodiments, the ammeter 116 may be a separate element.

For each viral particle 102 that passes through the nanopore 112, the circuit's resistance changes and which results in a measurable change in current amplitude. If the current amplitude is measured over time, the instances of viral particle 102 migration through the nanopore 112 can be observed and quantified. If, the exterior cavity 113 originally contained no viral particles 102, the number of viral particles that migrated through the nanopore 112 is the exact number of viral particles in the exterior cavity 113. Additionally, the rate of viral particle 102 migration can also be measured and be used to calculate the concentration of viral particles that remain in the interior cavity 106.

The number of events in the recorded ionic current may not perfectly match the translocated viral particle 102 count since smaller particle size or highly porous particles may not result in a detectable signal. In addition, collisions without translocation or migration can result in many consecutive current modulations that are not easily distinguishable. However, collision patterns are detectable and can be compensated for in post-processing of the measured current. In addition, existing methods, such as PCR for quantification of the total viral genome, may be used to estimate the number of viral particle that pass through the nanopore 112 during a calibration test and determine a correction factor for converting the number of current modulation events into the exact number of viral particles that transported the nanopore 112.

Isolated virus translocation nanopore experiments can also be utilized to maximize translocation efficiency and rate, pore shelf-life, experiment lifetime, and minimize clogging. The electronic signal or signature, comprising the modulation of amplitude over time during viral particle 102 migration, can also be used to provide information on the likely heterogeneity of characteristics (diameter, charge, dwell time, etc.) within variant viral particles as well as across different variants.

FIG. 1B is a graph 120 of current vs. time of the circuit created by the structure shown in FIG. 1A. The graph 120 has a y-axis 121 corresponding to the signal or current measured by the ammeter and an x-axis corresponding to the passage of time. Over time, the current varies as viral particles 102 migrate through the nanopore 112 and change the resistance of the circuit. This produces a measurable electronic signature 123, the circuit's current over time. Sudden magnitude changes or spikes, 124, correspond to a single virus particle 102 passing through the nanopore. The number of spikes 124 in electronic signature 123 denotes the number of viral particles 102 that passed through the nanopore 112 during the observed time period.

Referring again to FIG. 1A, after a known number of viral particles 102 pass through the nanopore 112 they enter the exterior cavity or chamber 113. The Chamber 113 is defined by the second structure 101 and is configured to contain a fluid 107b. After migration, fluid 107b will contain a known number of the viral particles 102. The second structure 101 includes a complimentary feature that is configured to interface with the shape 110 and removably couple with the first structure 100. When interfaced, the fluid 107b in the exterior cavity 113 is between the nanopore 112 and the sides of the second structure allowing for the flow of particles from interior cavity 106. The second structure 101 may include a surface 109 on which live cells 108 can adhere to. Alternatively, live cells may be suspended in fluid 107b or adhered to beads in the fluid 107b (not shown). The cells 108 can be infected by migrated viral particles 102 in the chamber 113 after a transduction time. As described previously, the number of viral particles 102 in the chamber 113, determined by the observation of the current modulation during migration, this count includes viral particles 102 that infect and/or associate with cells 108 and those that remain in the liquid 107b. It also does not discriminate between particles that have the functional surface proteins and the functional viral genome. Therefore, it cannot be used alone to determine infectivity of the viral particles 102. The cells 108 may be directly imaged from below the surface 109 using inverted, epifluorescence microscopy to quantify fluorescent cells (if using a fluorescent reporter such as green fluorescent protein (GFP) or red fluorescent protein (RFP). in the virus), smFISH may be performed on them directly to detect viral RNA particles in infected cells, or other known equivalent methods of determining infected cells. Based on the images of the cells 108 the number of infected cells can be determined. If the cells 108 are adhered to the wall 109, they may be imaged inside the cavity 113 of the second structure. If the cells 108 are suspended in liquid 107b, they may be removed from the second structure and then plated and imaged.

The infectivity of viral particles 102 can be determined based on the ratio between the number of viral particles in the cavity 113, determined with the first structure 100 based on the induced electronic signature, and the number of infected cells, determined by imaging the cells 108. For embodiments using smFISH is that the infectivity of natural/non-genetically engineered viruses may be determined directly. In such embodiments this can be done by controlling, using the nanopore 112 and manipulation the voltage gradient induced by the electrodes 104a, 104b, the number of viral particles 102 entering the cells 108 and the viral replication can be quantified (i.e., a single viral particle 102 passes through the nanopore 112 and infects a cell 108—how many RNA molecules are produced from that infection in a given period of time).

Some embodiments of the invention utilize a single-molecule FISH assay for the detection of AAV and lentivirus viral partilces 102 that infected the cells 108. In such embodiments, 20mer DNA oligonucleotide probes will be designed to target the constant region of the lentivirus and AAV vectors. The probes may be designed using the Stellaris probe design software, each labeled with a TEG-amino 3′ modification and obtained from Biosearch. Probes for each target are split into two groups, “odds” and “evens” whereby every other probe will be labeled with the same fluor. Individual probes from each set are combined and subsequently labeled using spectrally distinct, NHS-ester functionalized fluorophores, for non-limiting example Alexa 594 for the “evens” and Cyanine 5 for the “odds”. Fluorophore labeled probes can be purified using reverse-phase HPLC. While AAV or lentivirus viral particles 102 migrate through the nanopore 112 and the number of them in cavity 106 is determined based on the electronic signature comprising of measured current over time, the cells 108 on the surface 109 will be will be cross-linked with 4% paraformaldehyde and permeabilized and stored in 70% ethanol for each concentration condition every 8 hours for 48 hours until migration and transduction is complete. Embodiments of the invention can be used to determine the delivery rate of viral vectors used for chimeric antigen receptor (CAR) therapeutics (e.g., Car T-cells, CAR-NK cells, etc.), CRISPR therapeutics or vaccine delivery.

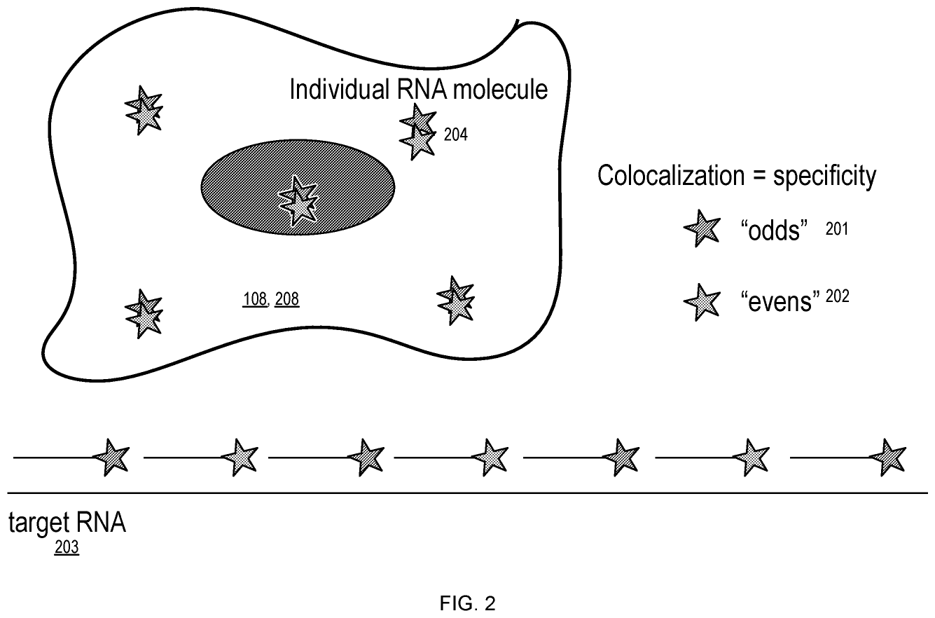

FIG. 2 is an illustration of an odds and evens single-molecule FISH labeling scheme. The odds 201 and evens probes 202 can be applied to each of the he crosslinked and permeabilized cell 108 samples. The cell samples 108 are then imaged and the spot counts of the probes 201, 202 can be quantified for each cell 202. A colocalization analysis for the odds and evens probes sets, colocalized spots 204 are strongly indicative of probe specificity because these spots are unlikely to randomly colocalize in the exact same XYZ coordinates. The probe sets can be combined by removing nonspecific probes from each set until we have achieved >90% colocalization in the two channels. The concentration of salt in the hybridization buffer and the percentage of formamide in the hybridization buffer and the washes can also be modified to maximize specificity as defined by colocalization percentage. Multiple fluorescent channels can be used to image the cell 208 samples to detect the EGFP protein expression and to detect each of the smFISH fluorescent spots. Raj lab image analysis pipeline may be used to determine the single-cell distribution of EGFP expression and compare this to the smFISH spot count single-cell distribution. As RNA expression precedes protein expression and it can take some time for individual RNA molecules to start translating into a protein, the FISH signal will pick up early viral infection prior to the EGFP expression. Finally, the number and percentage of cells that have colocalized smFISH signals can be calculated and compared these to the percentage of cells that have EGFP expression to determine sensitivity. Highly expressed RNA FISH spots that correspond to EGFP expression will help to validate that the probes are specifically labeling the desired mRNA construct. A lower limit of specific detection can be defined as the minimum number of spots necessary to call a cell positive and apply the formula for determining transducing units: TU/mL=(Number of cells transduced×Percent smFISH positive×Dilution Factor)/(Transduction Volume in mL).

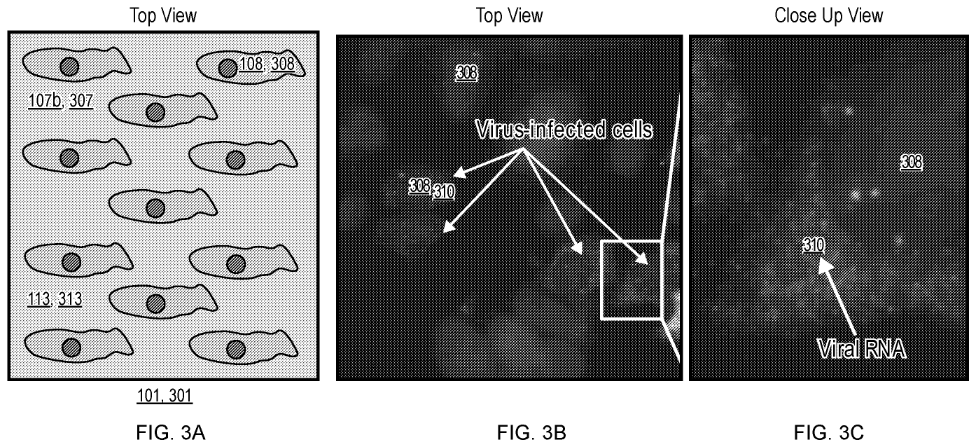

FIG. 3A is a top down view of the cell chamber, the second structure, 101, 301 containing the cells 108, 308. The live cells 108, 308 are adhered to the wall 109 of the second structure 101, 301. The second structure 101, 301 contains the fluid 107b, 307 in the exterior cavity 113, 313. The fluid 107b, 307 may be a cell culture medium. The exterior cavity 113, 313 receives the viral particles 102 transported through the nanopore 102 from the interior cavity 103 of the first structure 100 (not shown). The viral particles 102 mix with the fluid 107b, 307 and may infect the cells 108, 308.

FIG. 3B is an image of HeLa cells 308 infected with lentivirus. FIG. 3C is a closeup of an infected cell 308 in FIG. 3B. FIG. 3B is a top down image of a cell chamber containing cells 308. FIGS. 3B and 3C are taken with an imager than can be utilized by embodiments of the invention. The imaged cell chamber may be part of the second structure 101,301. A lentivirus may be introduced to the cell chamber through a nanopore from the first structure 100. The exact number of lentivirus in the cell chamber can be determined based on the induced electrical signature during migration through the nanopore 112. Lentivirus RNA 310 in infected cells can be detected by smFish and indicated a positive infection. The nuclei of the cells 30 can be DAPI stained to increase visibility. The Lentivirus RNA 310 may be stained with genome specific smFISH probes 201, 202 to allow it to be detected. The number of cells 308 infected can be determined by counting the number of cells 308 containing viral RNA 310. A person of ordinary skill in the art would understand that alternative methods of determine infected cells may be used instead of smFish imaging. Additionally, a person of ordinary skill in the art would understand that different viruses other than lentivirus may be analyzed with the methods discussed and shown herein. The number of cells that are actually infected with the viral particles can be determined using smFISH or equivalent methods. The electronic signature, created during viral particle 102 migration can be used to determine, the total number of viral particles in the chamber 113, 313 that have access to and the opportunity to infect the cells 108, 308. Therefore, infectivity can be calculated based on a ratio of the successfully infecting viral particle over the total viral particles migrated.

Embodiments of the invention may use an imager to detected nucleic acid, DNA and/or RNA that is either part of the migrated particles 102 or expression products, including proteins, of the migrated particles produced by the cells 108, 308. In additional, embodiments of the invention functional assays may be used instead of or in addition to imagers to detected and quantify nucleic acid.

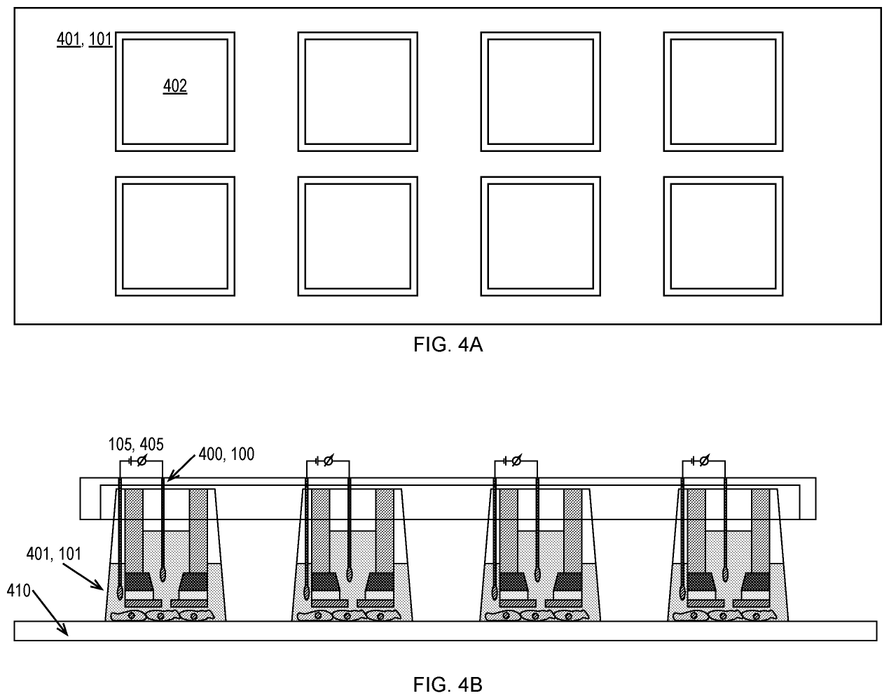

FIG. 4A is a top view of a currently existing cell culture tray 410. The cell culture tray 510 may be a commercially available chambered coverglass for cell culture and imaging. The cell culture tray 410 has cell culture chambers 401, 101 that can be used to grow cells in media. The cell chambers 401 of cell culture tray 410 can be the second structure 101 shown in FIG. 1A. The cell culture tray may include covers 402 for each of the cell culture chamber 401, 101. The cell culture tray 410 is widely used by many laboratories for virology and other experiments with utilizing live cells.

FIG. 4B is a side view of a cell culture tray 410 with a lid 100, 400 including a nanopore 112, in accordance with an example embodiment of the invention. The lid 100 may be modified to include an array of nanopores 112 for controlled injection and qualification of viruses and other agents or parties. In embodiments of the invention, the standard cell culture lid or lids 420 are replaced by one or more of the first structure 400, 100 (detailed in FIG. 1A). This modification allows the invention to be implemented on already commonly used and understood tool, the cell culture tray. The first structure 400, 100, with a nanopore chip 103 can be 3D printed to fit the cell chambers 401, 101 and serve as a replacement to the traditional lids 402 and interface with the cell chambers 401, 101 in the same manner using the same features. The 3D printer may be a form 2 stereolithography resin printer and use biomedical-grade resin to test for biocompatibility. The first structure 100, 400 maybe a modified chambered coverslip with a removable lid that includes the chamber 103 into which the viral sample 102 is injected. Such an embodiment, allows for viral particles to be deposited into the cell culture chambers 401, 101 and for the exact number of the deposited viral particles to be counted. This permits experiments, such as determining the number of infected cells, to be run on cells in the cells chambers 401, 101 while the exact number of viral particles in contact with those cells is known. In some embodiments, with multiple cell chambers 401, 101 and first structures 400, 100, each chamber may be used for different experiments with different viral particles, nanopore properties and/or cultured cells. The first structures, or modified lids, 400, 100 with nanopores may include mounting holes for Ag/AgCl electrodes 104a, 104b that come in contact with the electrolyte solutions 107a, 107b on both sides of the nanopore chip 103 and are connected to the power source 105, 405 and ammeter 116. The electrodes 104a, 104b are used to apply the voltage gradient and create the circuit used to induce and count the viral particles migrating into the cell chambers 401, 101. In some embodiments, each of the modified lids 400, 100 has a dedicated power source 105, 405. Alternatively, a single power source 105, 405 can be used to power electrodes 104a, 104b in multiple cell chambers 401, 101. Some embodiments may use Ag/AgCl electrodes adhered using injection molding it as a flexible PCB on polyimide or similar materials.

In some embodiments, to record the current modulations, a E4 miniaturized amplifier (Elements srl) with USB connectivity can be used to set the electrode voltage and record the ionic current of the created circuit. The amplifier device 405, 105 and the cell chamber tray 410 may be mounted inside a miniaturized faraday cage for noise reduction. The amplifier may also include control circuitry for clogging prevention, programming of desired particle counts, and real-time particle quality control. When coupled with a smFISH-based, or equivalent, infectious titer the determined viral particle count by can help establish a model for correlating the total count and characteristics of translocated virus particles with the total infections.

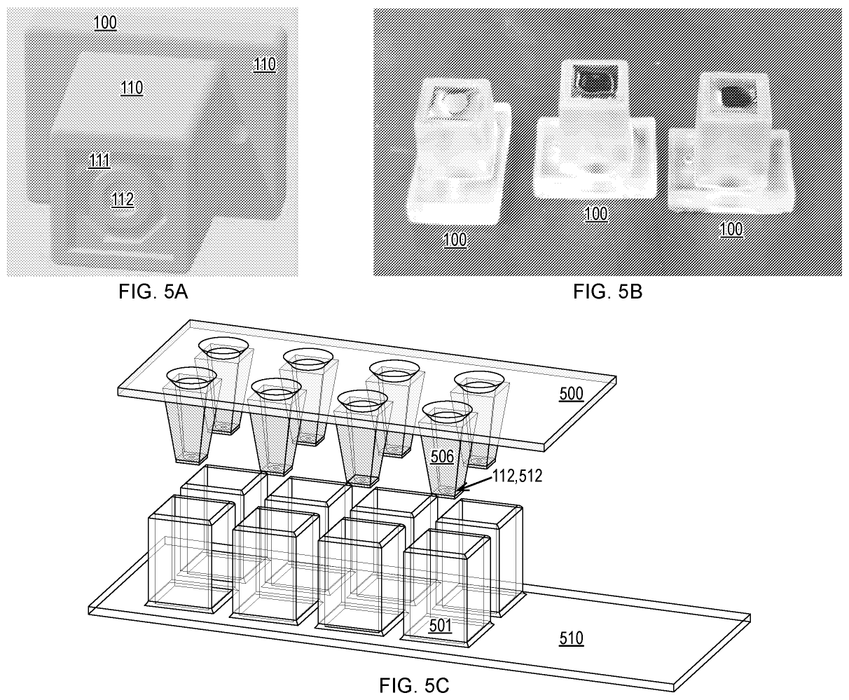

FIG. 5A is a rendering of the first structure 100 that includes a nanopore 112 utilized by embodiments of the invention. The first structure 100 include sides 110 and a barrier 111 that define an interior chamber 106 configured to hold a fluid 107a. The barrier 111 defines a nanopore 112 therethrough that allows for viral particles to migrate out of the interior chamber of the first structure 100. The first structure 100 includes shapes 110 configured to interface with complimentary features of a second structure, such as a cell chamber, that defines an exterior chamber into which the viral particles can migrate. FIG. 5B is a picture of 3D printed first structures 100 that include a nanopore utilized by embodiments of the invention and shown in the rendering of FIG. 5A. First structures 100 are able to provide a count of the viral particles that pass through nanopore 112 based on the observed change in amplitude over time.

FIG. 5C is an isometric diagram of a lid modified to include a nanopore and configured to interface with a set of cell culture chambers utilized by embodiments of the invention. The first structure 500, is a lid for the cell chambers 501 of the cell chamber tray 510. Each lid includes a nanopore 112, 512 through which viral particles can migrate. The first structure 500 has interior cavities 506 that will be fluidically coupled, via the nanopore 112, 512, when the first structure 500 is covering the cell chambers 501. The interior cavities 506 may have removable tops, or even no top, to allow for the depositing of fluids containing viral particles. For each interior cavity 506, structure 500 includes the necessary elements to (i) apply a voltage across its nanopore 112, 512, (e.g., electrodes 104a, 104b, and power source 105), (ii) measure the amplitude change during viral particle migration, and (iii) determine the number of viral particles that entered the corresponding cell culture chamber 501 using the methods and components described herein. Each of the cell culture chamber 501 may contain cells and enable the determination of the number of infected cells using the methods described herein. First structurer 500 may have different nanopore 112, 512 sizes that distribute particles into cell chambers 501 to allow different size particles to be examined in parallel.

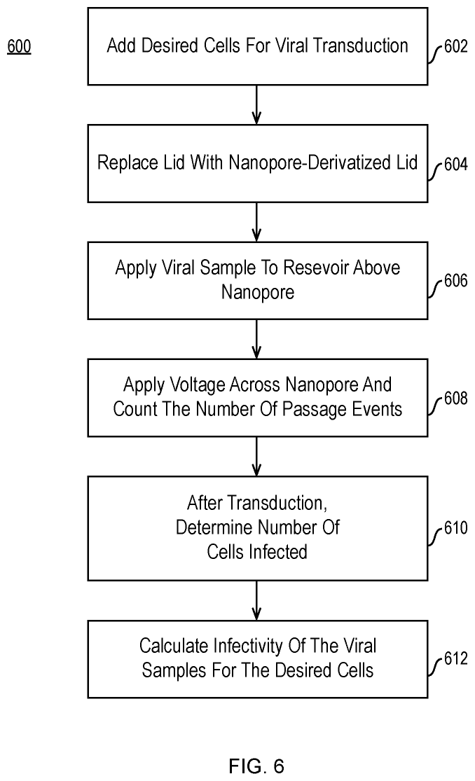

FIG. 6 is a flowchart of a method 600 for determining the infectivity of viral particles in an embodiment of the invention. Method 600 begins by adding 602 cells for viral transduction into a chamber. The chamber can be a cell culture chamber or chambered coverslip and the cells plated on a surface of that chamber. The chamber may be structures 101, 401 or 501 as detailed in FIGS. 1, 4 and 5C. The chamber can have a lid to prevent contamination while the cells are being plated or stored. After the cells achieve a desired level of confluency, the lid for the chamber is removed and replaced with a nanopore-derivatized lid in step 604. The nanopore-derivatized, or modified, lid may be structures 100, 400 or 500 as detailed in FIGS. 1, 4 and 5A-C. In step 604, the chamber and nanopore-derivatized lid may be connected together with complimentary shapes configured to interface.

In step 606, viral particles are added to a reservoir in the nanopore-derivatized lid above the nanopore. The reservoir is in fluid communication with the chamber containing the cells via the nanopore. In step 608, a voltage is applied across the nanopore creating a circuit with a path of ionic current from the reservoir through the nanopore and into the chamber. The voltage may be applied using electrodes as shown in FIG. 1. The voltage source may be incorporated into either the chambered coverslip or the nanopore-derivatized lid. Alternatively, the voltage source may be a separate device. While the voltage is applied, viral particles will migrate via the nanopore from the nanopore-derivatized lid reservoir and to the chamber containing the cells. As the migrate, they interrupt the ionic current flow via the nanopore altering the resistance of the circuit. This creates modulations in the current, that can be identified and defined as passage events. The current is observed and monitored and the number of passage events can be counted. The number of passage event corresponds to the number of migrated viral particles. If the cell chamber originally contained no viral particles, the number of migrated viral particles is the exact count of viral particles in the chamber. Other methods of quantifying viral particles can be used such as fluorescently labeling virions with surface protein specific antibodies to determine total counts as well as surface protein positive counts. This supplementary quantification methods can be used to confirm the number derived from the observed passage events and/or be used to determine a correction factor to better correlate the number of passage events with the number of migrated particles. When a desired number of viral particles have entered the cell chamber the voltage may be stopped or even reversed to prevent further migration. Upon completion of the migration and counting, the nanopore-derivatized lid can be removed from the cell chamber and the original lid returned.

In step 610, the cells are incubated for a desired transduction time and then the number of cells infected with viral particles is determined. This may be accomplished in several different ways such as, but not limited to, using viral particles that contain a fluorescent reporter or induce a fluorescent reporter in infected cells and measuring the number of fluorescent cells or crosslinking cells and using smFish to quantify viral infection directly and determine the number of infected cells and the single-cell viral RNA counts. Finally, in step 612, the infectivity of the viral particles can be determined using the total number of viral particles that had access to the cells, the count of migrated cells, during the transduction incubation time and the actual number of cells infected.

Embodiments of the invention may utilize client computer(s)/devices and server computer(s) that provide processing, storage, calculation functions, and input/output devices executing application programs and the like. Client computer(s)/devices can also be linked through communications network to other computing devices, including other client devices/processes and server computer(s). Communications network can be part of a remote access network, a global network (e.g., the Internet), cloud computing servers or service, a worldwide collection of computers, Local area or Wide area networks, and gateways that currently use respective protocols (TCP/IP, Bluetooth, etc.) to communicate with one another. Other electronic device/computer network architectures are suitable. Client computers/devices and server computer(s) may execute any of the computation steps or processes of embodiments of the invention including method 600, the analyzing of electronic signatures, counting of particles, determining infectivity, displaying and cell images, and/or controlling power source 105, electrodes 104a, 104b and other components of embodiments of the invention disclosed herein. Client computers/devices and server computer(s) may include memory that provides volatile storage for computer software instructions and data used to implement method 600 and other embodiments and/or elements of the present invention. Client computers/devices and server computer(s) may include central processor units, connected the memory by a bus, that provides for the execution of the computer instructions to implement method 600 and other embodiments and/or elements of the present invention. Central processor units may work alone or in combination implement method 600 and other embodiments and/or elements of the present invention. Client computers/devices and server computer(s) may also be utilized to control components of embodiments the invention such as power source 105, ammeter 116, detectors, and imagers.

The computation steps or processes of embodiments of the invention may be a included in a computer program product, including a computer readable medium (e.g., a removable storage medium such as one or more DVD-ROM's, CD-ROM's, diskettes, tapes, etc.) that provides at least a portion of the software instructions for the invention system. The computer program product can be installed by any suitable software installation procedure, as is well known in the art. In another embodiment, at least a portion of the software instructions may also be downloaded over a cable, communication and/or wireless connection.

The invention also includes and enables methods for assessing efficiency of particles. In one embodiment, the method comprises applying a voltage gradient across a nanopore to induce particles to migrate from a first cavity to a second cavity via the nanopore to produce an electronic signature representative of each particle of migrated particles, the second cavity containing cells; counting a number of particles based on the electronic signature to create a total particle count of migrated particles; determining a number of cells with which at least a subset of the migrated particles associated to produce a total associated cell count; and determining efficiency of the particles based on a ratio of the total associated cell count and the total particle count.

Another aspect of the invention includes and enables a method of assessing the efficacy of an anti-microbial (e.g., anti-viral) agent (or a vaccine, e.g., an anti-microbial (such as anti-viral) vaccine). In one embodiment, the method comprises contacting a sample of cells with an anti-viral agent or vaccine to produce a test sample; exposing the test sample to a specific number of viral particles by controlling a flow of viral particles to the test sample via an electric field applied to a nanopore; and comparing a percentage of cells in the test sample infected by the viral particles to a reference standard. In some embodiments, the test sample will have a fluorescent reporter delivered to it by the particles. The fluorescent reporter may be a green fluorescent protein (GFP) or red fluorescent protein (RFP). In some embodiments if a lower percentage of infected cells in the test sample than in the reference standard is indicative of efficacy of the anti-viral agent or anti-viral vaccine. The reference standard may be fine a background level of detection.

In some embodiments, the particles or viral particles analyzed and quantified can bind to cell receptors. For a non-limiting example, the particles may be lentiviruses with VSV-G to infect human cell lines and/or the particles may be SARS CoV-2 and the cells are from an ACE2 cell line

In some embodiments, the sample is a biological sample. As used herein, “biological sample” refers to any sample that can be from or derived from a subject, e.g., a human subject. The methods disclosed herein can be performed using a variety of possible biological sample types. For example, a cell lysate, a population of cells, a cell culture, a tissue, or a biological fluid. In some embodiments, a “biological sample” refers to a collection of similar fluids, cells, or tissues isolated from a subject, as well as fluids, cells, or tissues present within a subject. Samples include, but are not limited to, samples that are biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, synovial fluid, liquid solutions contacted with a subject or biological source, for example, cell and organ culture medium including cell or organ conditioned medium, lavage fluids, tissue biopsies, tumor tissue biopsies, tumor tissue samples, fine needle aspirations, surgically resected tissue, organ cultures or cell cultures. In some embodiments, the biological sample is a bodily fluid sample), a nasal (e.g., nasal swab) sample, buccal (e.g., buccal swab) sample, a hair sample (e.g., from hair follicles or a skin sample. Non-limiting examples of biological fluids (bodily fluids) include blood (e.g., whole blood and derivatives and fractions of blood, such as plasma or serum), bone marrow aspirates, cerebrospinal fluid, extracted galls, GCF gingival crevicular fluid, milk, prostate fluid, pus, saliva (including whole saliva, individual gland secretions, oral rinse), skin scrapes, sputum, surface washings, tears (liquid secreted by lacrimal glands), and urine. In certain embodiments, the bodily fluid comprises blood, saliva, sputum, tears, urine or semen, or a combination thereof. In some embodiments, the bodily fluid comprises white blood cells.

In some embodiments, the percentage of cells associated with a particle (e.g., infected with a viral particle) in the sample is compared to that in a control sample or a reference standard.

In some embodiments, the control sample comprises a biological sample from an unaffected subject or a population of unaffected subjects. An unaffected subject is a healthy subject or a subject not exposed to a particle (e.g., a viral particle).

In some embodiments, the methods provide for assessment and/or selection of an antimicrobial agent (e.g., an anti-viral agent). In some embodiments, the control sample comprises a biological sample from an untreated subject (e.g., a subject who has not been administered an anti-viral agent, e.g., an anti-viral drug). In some embodiments, the control sample comprises a biological sample from an unvaccinated subject (e.g., a subject who has not been administered a vaccine, e.g., anti-viral vaccine). In some embodiments, the control sample is a cell line, wherein the cells do not have a receptor which binds to the particle.

The term “reference standard” can be, for example, a mean, an average, a numerical mean or range of numerical means, a pattern or a corresponding measurement or assessment derived from a reference subject (e.g., an unaffected or untreated or unvaccinated subject) or reference population (e.g., a population of unaffected or untreated or unvaccinated subjects).

In some embodiments, the control sample is from a sample from a mammal, e.g., a human subject, e.g., from an age-matched subject, or a subject matched based on relevant criteria. In certain embodiments, the control sample is a theoretical value calculated from the general population. In particular embodiments, the control sample is a baseline sample of the subject, e.g., before treatment and/or exposure to the particles or agent (e.g., anti-viral agent or vaccine, such as an anti-viral vaccine).

The terms “subject” and “patient” are used interchangeably herein. “Patient in need thereof” or “subject in need thereof” refers to a mammalian subject, preferably human, diagnosed with or suspected of having a disease or disorder, e.g., a viral disease or disorder, who will be or has been administered an anti-viral agent or a viral vector according to a method of the invention. “Patient in need thereof” or “subject in need thereof” includes those subjects already with the undesired physiological change or disease well as those subjects prone to have the physiological change or disease.

“Treat,” “treating” or “treatment” refers to therapeutic treatment wherein the objective is to slow down (lessen) an undesired physiological change or disease, or to provide a beneficial or desired clinical outcome during treatment. Beneficial or desired clinical outcomes include alleviation of symptoms, diminishment of extent of disease, stabilized (i.e., not worsening) state of disease, delay or slowing of disease progression, amelioration or palliation of the disease state, disease remission (whether partial or total, whether detectable or undetectable), and prolonging survival as compared to expected survival if a subject was not receiving treatment or was receiving another treatment.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A system for assessing properties of particles, the system comprising:
a first structure defining an interior cavity, configured to contain an interior cavity fluid, and a barrier separating the interior cavity from an exterior cavity configured to contain an exterior cavity fluid, the barrier defining a nanopore therethrough fluidically coupling the interior and exterior cavities, the first structure further comprising at least one feature configured to interface with at least one complementary feature of a second structure that defines the exterior cavity; and
electrodes configured to energize and apply a voltage gradient across the nanopore between the interior cavity and exterior cavity, the voltage gradient of sufficient magnitude to induce particles to migrate via the nanopore between the interior cavity and the exterior cavity and produce an electronic signature representative of at least one property of the particles.

2. The system of claim 1 further comprising:
a detector configured to observe the electronic signature; and
a processor configured to determine a number of particles that migrated via the nanopore based on the electronic signature observed.

3. The system of claim 1 further comprising:
the second structure, wherein the second structure defines a wall that is separated from the barrier with the exterior cavity fluid therebetween while the at least one feature of the first structure is interfaced with the at least one complementary feature of the second structure.

4. The system of claim 3 further comprising an imager configured to determine a number of cells in the second structure to which at least a subset of the particles associated themselves, the imager further configured to provide a representation of the number of cells determined to the processor.

5. The system of claim 4 wherein the particles are viral particles and wherein the processor is further configured to determine infectivity of the particles based on a ratio of the number of particles that migrated via the nanopore and the representation of the number of cells determined to have at least one viral particle associated therewith.

6. The system of claim 4 wherein the imager is an epifluorescence microscope and the particles include a fluorescent reporter detectable by the epifluorescence microscope.

7. The system of claim 4 wherein the imager is configured to detect nucleic acid of the particles or expression products of the particles.

8. The system of claim 1 wherein the particles are microbes.

9. The system of claim 1 wherein particles are vectors.

10. The system of claim 9 wherein the vectors provide nucleic acid to the cells.

11. The system of claim 5 wherein the processor is further configured to determine a particle size distribution.

12. The system of claim 3 wherein the second structure defines a microfluidic channel in fluidic communication with the nanopore.

13. The system of claim 2, wherein the second structure is a cell culture chamber and wherein the system further comprises a cover configured to removably couple with the second structure by interfacing with the least one complementary feature of the second structure and wherein the first structure is a lid, substitutable for the cover.

14. The system of claim 1 further comprising:
a detector configured to observe the electronic signature; and
a processor configured to determine a number of particles that migrated via the nanopore based on the observed electronic signature and to assess a number of particles that are less than about 1 micrometer in size.

15. A method for assessing properties of particles, the method comprising:
interfacing a first structure to a second structure by way of coupling at least one mechanical complementary feature of the first structure to at least one mechanical complementary feature of the second structure, the first structure defining an interior cavity, the first structure further defining a barrier separating the interior cavity from an exterior cavity configured to contain an exterior cavity fluid, the barrier containing a channel structure defining a nanopore therethrough fluidically coupling the interior and exterior cavities, the first structure further comprising at least one feature defining a shape configured to interface with at least one complementary feature of a second structure that defines the exterior cavity; and
applying a voltage gradient across the nanopore between the interior cavity and exterior cavity by energizing electrodes, the voltage gradient being of sufficient magnitude to induce particles to migrate via the nanopore between the interior cavity fluid and the exterior cavity fluid and to enable observing of an electronic signature representative of at least one property of the particles produced by passing through the nanopore.

16. The method of claim 15 further comprising adding an interior cavity fluid containing the particles to the interior cavity.

17. The method of claim 15 further comprising:
observing, using a detector, the electronic signature to produce an observed electronic signature; and
determining, using a processor, a number of particles that migrated via the nanopore based on the observed electronic signature.

18. The method of claim 17 further comprising:
determining, with an imager, a number of cells in the exterior cavity of the second structure, with which at least one particle associated; and
determining, using a processor, the infectivity of the particles based on a ratio of the number of particles that migrated via the nanopore and the number of cells with which at least one particle associated.

19. The method of claim 15 wherein the particles are microbes.

20. A method for assessing efficiency of particles in a sample comprising cells, the method comprising:
applying a voltage gradient across a nanopore to induce particles to migrate from a first cavity to a second cavity via the nanopore to produce an electronic signature representative of each particle during migration, the second cavity containing cells;
counting a number of particles based on the electronic signature to create a total particle count of migrated particles;
determining a number of cells with which at least one of the migrated particles associated itself to produce a total associated cell count in the second cavity; and
determining efficiency of the particles based on a ratio of the total associated cell count and the total particle count.

21. A method of assessing the efficacy of an anti-viral agent, the method comprising:
contacting a sample of cells with an anti-viral agent to produce a test sample;
exposing the test sample to a specific number of viral particles by controlling a flow of viral particles to the test sample via an electric field applied to a nanopore, the viral particle originating from a first cavity defined by first structure and the test sample is in a second cavity defined by a second structure, the first cavity and the second cavity in fluid communication via the nanopore;
determining a percentage of cells in the test sample infected by the viral particles in the second cavity; and
comparing the percentage of cells in the test sample infected by the viral particles to a reference standard to determine efficacy of the anti-viral agent.

22. The method according to claim 21 wherein the reference standard and the test sample are from the same subject.

23. The method of claim 15, wherein the particles or viral particles that can bind to cell receptors.

24. The method of claim 15, further comprising applying single-molecule RNA fluorescence in situ hybridization (smFISH) to determine a percentage of cells infected with the particles or viral particle.

25. The method of claim 15, further comprising imaging a fluorescent reporter delivered to the test sample.
